(19) United States
(12) Patent Application Publication
(10) Pub. No.: US 2014/0271927 A1
(43) Pub. Date: Sep. 18, 2014

(54) COMPOSITION AND METHOD FOR TREATING NEUROPATHY

(71) Applicant: Agustin Andrade, Miami, FL (US)

(72) Inventor: Agustin Andrade, Miami, FL (US)

(21) Appl. No.: 14/208,576

(22) Filed: Mar. 13, 2014

Related U.S. Application Data
(60) Provisional application No. 61/791,102, filed on Mar. 15, 2013.

(51) Int. Cl. A61K 33/04; A61K 33/18; A61K 31/4402; A61K 45/06; A61K 31/385; A61K 31/202

(52) U.S. Cl. CPC A61K 33/04; A61K 31/385; A61K 31/202; A61K 31/4402; A61K 45/06; A61K 33/18
USPC 424/667; 424/702

(57) ABSTRACT

The present invention provides therapeutic and nutrient compositions comprising Alpha lipoic acid, gammalinolenic acid, chromium, selenium, and optionally iodine for the treatment of diabetic neuropaty. These compositions ameliorate the condition and symptoms associated with the complications of lack of triiodothyronine in the nucleus of target cells, in particular and the pathological effects of the vaso nervorum.

### CROSS REFERENCE AND CLAIM OF PRIORITY

This application claims priority to U.S. provisional patent application No. 61/791,102 filed on Mar. 15, 2013, the contents of which are expressly incorporated by reference. All references cited herein are expressly incorporated by reference.

### FIELD OF INVENTION

The present invention relates to compositions for and methods of treating diabetic neuropathy using alpha lipoic acid, gammalinolenic acid, chromium, iodine, cinnamon and Selenium.

### BACKGROUND OF THE INVENTION

There are approximately 20 million people with Diabetes Mellitus in the United States and 189 million world-wide. The incidence of diabetic neuropathy is present in approximately 40 to 60% of the entire diabetic population and 60% are symptomatic.

The pathogenesis of the neuropathy is a microscopic vasculitis and subsequent ischemia or infarction of the nerve. Focal ischemia results in segmental Demyelination followed by remyelination. In diabetic patients, re-myelination is defective and delays the repair of focal deficits. Delayed remyelination may be a consequence of diabetes-induced Schwann cell dysfunction. (1)

In the case of the Distal Symmetrical polyneuropathy (DSPN) there are several hypotheses. The prevailing theory is that persistent hyperglycemia is the primary factor. The gluco-toxicity may cause a pathway, lipid metabolism, deficiencies of dihomogammalinolenic acid (GLA) and carnitine glycation or AGE formation. It increases oxidative stress and growth factor defects.

Histopathologic studies show the presence of different degrees of endoneuria and epineuria microvasculopathy, mainly vessel basement membrane thickening and obstruction of vasa nervorum.

One of the complications of diabetes neuropathy is neuropathic foot ulceration, 83% of the 120,000 non-traumatic lower extremity amputation performed each year in the United States, involve persons with diabetes (2). Post amputation mortality rates associated with diabetes are worse than cancer, ranging from 13 to 40% after 1 year and 35 to 65% after 3 years (3).

There is no treatment per se for neuropathy except for partial improvement of painful neuropathy. Available treatments include triclyclic antidepressants, anticonvulsants, and selective serotonin reuptake inhibitors (“SSRI”). All of these medications have well documented undesirable side effects.

Alpha lipoic acid (Thioctic acid), a derivative of cationic acid, is present in food and is also synthesized in the liver and, is a natural cofactor in the pyruvate dehydrogenase. It has proved effective in ameliorating the somatic and autonomic neuropathies in Diabetes Mellitus.

Gamma Linolenic acid is an essential fatty acid that is metabolized to GLA, which serve as important constituent of neuronal membrane phospholipids and also a substrate for prostaglandin formation, seemingly important for preservation of nerve blood flow. In diabetic patients conversion of gamma linoleic acid to gammalinolenic acid and subsequent metabolite is impaired, this substance has demonstrated significant improvements in both clinical and electrophysiological tests.

### SUMMARY OF THE INVENTION

The inventor has surprisingly identified that thyroid problems play a significant role in diabetic neuropathy.

The prevention of hypothyroidism in Diabetic Patients has not being targeted yet in an effort to maximize the reduction of cholesterol, and improvement of the Hypotension, diastolic hypotension, impaired endothelium-mediated vasodilatation, elevation of the CRP (C-reactive protein) and elevation of homocystein which are very important risk factors in Diabetic patients. There are 27 million people with thyroid disease in United States. 3.7% of the Americans have hypothyroidism, the combination of hypothyroidism with Diabetes is a dreadful.

This invention relates to the use of nutrients and therapeutic composition to ameliorate disease condition, symptoms and disorders resulting at least in part from lack of production of Prostaglandin E and a decrease in production of LDL receptors.

The nutrients and therapeutical composition of this invention are useful in the prevention and treatment of symptoms and disease condition associated with diabetic complication including macroangiopathy and microangiopathy causing neuropathy and cardiovascular disorders and prevent progression of complication related to the poor function of the triiodothyronine in the nucleus of target cells, which causes morbidity that will increase in the development of cardiovascular disease.

In a specific embodiment of the present invention provides therapeutic and nutrient compositions and treatment methods using those compositions for amelioration condition and symptoms associated with the complications of lack of triiodothyronine In the nucleus of target cells in particular and the pathological effects of the vaso nervorum.

In one embodiment formula comprises Alpha lipoic acid, gammalinolenic acid, chromium and selenium. Iodine is optionally present.

### DESCRIPTION OF THE FIGURE

FIG. 1 is a bar graph showing the efficacy of the formulations of the present invention.

### DETAILED DESCRIPTION OF THE INVENTION

The nutrient and therapeutic composition of this invention are generally directed toward the improvement of the disease conditions where the thyroid subclinical hypothyroidism and or hypothyroidism are related to Diabetes and cardiovascular disease.

Elevation of total cholesterol and low-density lipoprotein cholesterol (LDL C) occurs in hypothyroidism due to increases the expression of cell surface LDL-C receptor expressed in fibroblast, liver and other tissues, this is mediated by STEROL REGULATORY ELEMENT-BINDING PROTEIN-2(SREBP-2). This protein is regulated by triiodothyronine. In addition there may be an increase of absorption of cholesterol in the gut.

Cholesteryl ester transfer protein (CETP) is decreased in hypothyroidism this protein transfers cholesterol from HDL_C to LDL-C and VLDL. In addition there is a change in the hepatic lipase, this will cause a change in the subfractions of HDL-C (Nascent, HDL2 and HDL3). Finally thyroid hormone increases the efflux from macrophages to HDL, via the ATP binding cassette Al.

The thyroid gland contains the highest selenium concentration and is considered to have substantial structural and functional role in glutathione peroxidase, iodothyronine deiodinases, thioredoxin reductases, all of which are selenoprotein. In addition, selenium works as anti-oxidant. It is known that even if the level of plasma selenium is normal, does not guaranty normal tissue level in the thyroid. Studies with selenium decreased the TPO antibodies titers and the well-being in general.

Thyroid hormones are known are known to regulate protein synthesis by acting at the transcriptional level and inducing the expression of many genes in bench studies it was found that T3 treatment to INS-1E cells increases insulin expression (Thyroid Vol 22; number 2, 2012). Symptoms that are associated with degeneration of the vaso-nervorum in diabetic patients or other entities where the damage of the vasonervorum either directly or indirectly affect the myelin of the axon and therefore it caused an alteration of the electrical conduction also called neuropathy and prevention or treatment of the autoimmune thyroid disease or in certain environment a deficiency of iodine as a cause of thyroid function deficiency.

Diabetic neuropathy is divided in peripheral neuropathy and autonomic neuropathy. This condition is at least secondary to the lack of prostaglandin E. the neuropathy autonomic is an independent risk factor for mortality and cardiovascular disease.

Diabetic peripheral neuropathy is the primary cause of the amputation of limbs in United States.

The main cause of mortality in Diabetic Patients is the cardio vascular disease, due to atherosclerosis and fragility of the microvasculature and microvasculature. The main treatment for this is the perfect treatment of the lipids and blood pressure and perhaps albeit unlikely the glycemia control itself.

I surprisingly discovered that by decreasing the autoimmune process of Hashimoto's and or giving iodine when there is iodine deficiency there is a consequent improvement of the function of the thyroid that provides the necessary T4 to be converted by deiodinase to T3 this will enable the production of LDL receptors, so there is no building up of this small lipoprotein which is well known to be an independent risk factor for cardiovascular disease in Diabetic patients.

In another twist Selenium has been successfully used to treat inflammatory disease U.S. PATENT 2007/0026090A1. Selenium being an inmmunomodulator will decrease complement Fixing anti-bodies and antibodies to gangliosides (anti-GM1) which may have a role in the pathogenesis of somatic neuropathy.

The treatment method described herein employ formulation to improve the electrical conduction measured by bio-thesiometer, improve the symptoms of the patient and decrease the LDL cholesterol by ensuring enough T3 in the cytoplasm for proper production of LDL receptors and therefore proper clearance of the small lipoproteins which will confer to the Diabetic patient not only a decrease of the symptoms of neuropathy and by improving the electrical condition will probably decrease the risk of amputations.

The first component is alpha lipoic acid (Thioctic acid), a derivative of octanoic acid, is present in food and is also synthesized in the liver and, is a natural cofactor in the pyruvate dehydrogenase. It has proved effective in ameliorating the somatic and autonomic neuropathies in diabetes mellitus. It is preferred to administer the alpha lipoic acid in an amount from 200-800 mg/day.

The second component is Gamma linolenic acid is an essential fatty acid that is metabolized to dihomo-γ-linolenic acid (DGLA), which serve as important constituent of neuronal membrane phospholipids and also a substrate for prostaglandin formation, seemingly important for preservation of nerve blood flow. In diabetic patients conversion of linoleic acid to gammalinolenic acid and subsequent metabolite is impaired, this substance has demonstrated significant improvements in both clinical and electrophysiological tests. It is preferred to administer the gamma linolenic acid in an amount from 100-500 mg/day.

The third component, selenium, is considered to have substantial structural and functional role in glutathione peroxidase. iodothyronine deiodinases, thioredoxin reductases, all of which are selenoproteins. In addition, selenium works as anti-oxidant. It is known that even if the level of plasma selenium is normal, does not guaranty normal tissue level in the thyroid. Studies with selenium decreased the TPO antibodies titers and the well-being in general. See Konstantinos A. Toulis, et al., Selenium Supplementation in the Treatment of Hashimotos Thyrdoiditis: A systematic Review and a Meta-anlaysis, Thyroid, Vol 20: 10 p 1163 (2010). It is preferred to administer the selenium in an amount from 60-200 mcg/day.

The forth component is chromium histidine or chromium picolinate. Chromium deficiency has been associated to with hyperglycemia, insulin resistance, hyperlipidemia, hypertension and increased risk for diabetes and cardiovascular disease, chromium levels decreases with age, glucocorticoid therapy, critical illness and with diets rich in concentrated sweets. Unfortunately its blood concentration may not adequately reflect total body stores. A series of published studies were reviewed which suggested a beneficial effect of chromium on carbohydrate metabolism, with several suggesting an insulin sensitizing effect glucose control can improve 1-2% lowering the HbgAlc reported. See William T. CEfalu and Frank B. Hu, Role of Chromium in Human Health and Diabetes, Diabetes Care, Vol 27:11 p2741 (2004). It is preferred to administer the chromium picolinate in an amount from 200-600 mcg/day.

An optional fifth component is iodine. Due to low salt diets imposed on patients to treat high blood pressure, many people do not get adequate iodine to maintain thyroid function. The U.S. RDA for iodine is 150 mcg.day. Iodine may be administered as potassium iodide in an amount from about 100 mcg/day to about 200 mcg/day and is most preferably administered at 150 mcg per day.

Any food or pharmaceutical grade of the above agents may be used. The compositions may be delivered individually or are preferably combined for ease of administration. The dosages can be given in one dose or may be administered in divided doses two, three or four times daily. The product can be delivered as a liquid or processes into soft gel capsules using techniques well known in the art.

One of skill in the art will appreciate that additional dietary supplements or vitamins can be added to the present formulation if desired. The present invention could be incorporated into a multivitamin composition or in food products. Known delivery systems for fatty acid supplements are likely to be effective with this invention.

Treatment should be maintained for as long as the neuropathy is present or until treatment is stopped by a physician.

### Example 1

Table 1 below shows an embodiment of the present invention.

TABLE 1

| Ingredient | Daily Dosing |
|---|---|
| Alpha lipoic acid | 500 mg |
| Gamma linolenic acid | 200 mg |
| Chromium picolinate | 200 mcg |
| Selenium | 200 mcg |

The above ingredients are place in a suitable vessel and mixed. Because the alpha lipoic acid and the gamma linolenic acid are in liquid oil form the resulting product is an oil based suspension of chromium picolinate and selenium. The resulting suspension can be administered as is or can be processed in soft gel capsules or further processed with other excipients to yield tablets. Optionally 200 mcg of iodine may be administered.

The above daily dosing is preferably divided into dosing units comprising one half the above daily dose such that each dose comprises the following

TABLE 2

| Ingredient | Daily Dosing |
|---|---|
| Alpha lipoic acid | 250 mg |
| Gamma linolenic acid | 100 mg |
| Chromium picolinate | 100 mcg |
| Selenium | 100 mcg |

100 mcg of iodine may be optionally added to the formulation of table 2. The above dose would be taken twice daily by patients with moderate neuropathy and would be taken three or even four times a day for severe neuropathy.

Efficacy can be tested in a controlled study as follows. For a period of 4 weeks, 10 patients will be assigned in open label to placebo and 10 to the above formulation. Neuropathic symptoms (4) will be scored at baseline and day 28 as total Diabetes Nuropathy Symptom score (DNS) and measurements with biothesiomether. The placebo patients will also received a combination of Folic Acid with Vitamin B6 and B12. Results will be analyzed using the Chi Square with one degree of freedom.

### Example 2

For a period of 4 weeks, 10 patients were assigned in open label to placebo and 10 to alphalipoic 500 mg and Borage Oil 500 mg equal to 200 mg of gamma linolenic acid (GLA) this combination is marketed as (DHV). Neuropathic symptoms (4) were scored at baseline and day 28 as total Diabetes neuropathy symptom score (DNS) and measurements with biothesiomether. The placebo patient also received a combination of Folic Acid with Vitamin B6 and B12. Results were analyzed using the Chi Square with one degree of freedom.

The total DNS at baseline for the Placebo patients was 83 and the score for patients assigned DHV was 86. At the re-assessment in 4 weeks the total score for Placebo and DHV was 90 and 41 respectively, this is a 55% decrease in symptoms with a P: <0.01. The average Diabetic Neuropathic score was 8.3 in the placebo and the DHV assigned group 8.6 at baseline screening. Four weeks later the average in the placebo patients was 9.0 whereas in the DHV patient was 4.1 points. See FIG. 1

In the Biothesiomether measurement there was a combined patient score at baseline of 401 in the patients allocated to placebo and 414 in the patients allocated to study composition. Four weeks after, the patient-score allocated to placebo and study composition was 405 and 343 respectively, this significant 15% improvement in the threshold for vibratory sensation with a P: <0.05. The average measurement in biothesiomether was in the placebo patients 40.1 and in the DHV assigned patient was 40.4 but after 4 weeks the average measurement in the placebo patient was 40.5 whereas in the DHV assigned was 34.3

One of skill in the art that other agents can be added to the present composition without substantially changing its properties to treat diabetic neuropathy, including the addition of other nutrients, task masking agents and excipients for formulation.

### REFERENCES

All references cited herein are expressly incorporated in their entirety by reference.

1. Williams's textbook of Endocrinology tenth edition 1542-1543.

2. Moaz Mojaddidi; Current diabetes report Vol. 5 number 6; 423-427.

3. Reiver GE; epidemiology of foot ulcers and amputation in the diabetic foot. The diabetic foot Mosby; 2001; 13-32.

4. Meijer JWG, Diabic Neuropathic Score; Diabetes Medicine, 19:962-5.

5. Ametov A; the SYDNEY Study. The sensory symptoms of diabetic polyneuropathy are improved with alphalipoic acid. Diabetes Care 2003; 26; 770-776.

6. Ziegler D treatment of symptomatic diabetic polyneuropathy with the antioxidant (ALADIN III). Diabetes Care 22; 1999; 1296-1301.

7. Ziegler(Deutche Kardiale Autonome Neuropathy). Diabetes Care 20;369-373,1997.

1. A composition for treating diabetic neuropathy comprising a dosage form comprising Alpha Lipoic Acid, Gamma Linolenic Acid, Chromium Picolinate and Selenium.

2. The composition of claim 1 where in the dosage form comprises from about 200 to about 800 mg of alpha lipoic acid, from about 100 to about 500 mg of gamma linolenic acid, from about 200 to about 600 mcg of chromium picolinate and from about 60 to about 200 mcg of selenium.

3. The composition of claim 2 wherein the dosage form comprises 250 mg of alpha lipoic acid, from 100 mg of gamma linolenic acid, from 100 mcg of chromium picolinate and 100 mcg of selenium.

4. A method of treating diabetic neuropathy comprising protecting thyroid hormones from autoimmune attack.

5. The method of claim 4 wherein the selenium is administered to protect the thyroid hormones from autoimmune attack.

7. The method of claim 5 wherein the patient receives from about 60 to about 200 mcg/day of selenium.

8. A method of treating diabetic neuropathy according to claim 1 further comprising the administration of at least one agent to promote thyroid health.

9. The method of claim 8 wherein the chromium is administered to promote thyroid health

10. The method of claim 5 wherein the patient receives from about 200 to about 600 mcg/day of chromium picolinate.

11. A method of treating diabetic neuropathy comprising the administration of at least one fatty acid in combination with at least one modulator of thyroid activity.

12. The method of claim 5 where the patient receives about 200 to about 800 mg/day of alpha lipoic acid, from about 100 to about 500 mg/day of gamma linolenic acid, from about 200 to about 600 mcg/day of chromium picolinate and from about 60 to about 200 mcg/day of selenium

13. The method of claim 12 further comprising the administration of iodine from 90 mcg/day to about 30 mg/day.

14. The composition of claim 1 further comprising iodine.

15. The composition of claim 2 further comprising from 100 mcg to about 200 mcg of iodine.

16. The composition of claim 3 further comprising 150 mcg iodine.
